(19) United States
(12) Patent Application Publication
(10) Pub. No.: US 2010/0323435 A1
(43) Pub. Date: Dec. 23, 2010

(54) Injection System for Bio-fuel Reactor

(76) Inventors: Akwasi A. Boateng, Royersford, PA (US); Neil M. Goldberg, North Wales, PA (US)

Correspondence Address: USDA, ARS, OTT, 5601 SUNNYSIDE AVE, RM 4-1159, BELTSVILLE, MD, 20705-5131, US

(21) Appl. No.: 12/823,319

(22) Filed: Jun. 25, 2010

(51) Int. Cl. C12M 3/00; C10B 1/00; C10J 3/00; B01J 8/18; G05D 7/00

(52) U.S. Cl. 435/289.1; 202/105; 48/111; 422/139; 137/1

(57) ABSTRACT

The bio-fuel reactor injection system includes supplementing a biomass feedstock with a feedstock gas so that thermal conditions within the reactor body are optimized. The feedstock gas facilitates the flow of the feedstock through the feedstock injection system and maintains the feedstock below the feedstock's melting point until the feedstock is injected into the reactor body. In the preferred embodiment, the supplemental feedstock gas is nitrogen. The injection system also includes a plurality of screens that form a gas distributor plate. The gas distributor plate at least partially supports a fluidized bed within the reactor body. In the preferred embodiment, the nitrogen-supplemented feedstock is injected into the fluidized bed within the reactor.

### FIELD OF THE INVENTION

The present invention relates to a feedstock and fluidization gas injection system for a reactor for the production of advanced bio-fuels. Specifically, the current invention relates to a lignocellulosic biomass feedstock injection system and a gas distributor plate system for a fast pyrolysis or a gasification reactor system.

### BACKGROUND OF THE INVENTION

The rising cost and shrinking supply of fossil fuel-based energy is driving an expanding interest in bio-based renewable energy. One source of renewable energy is agriculturally generated lignocellulosic biomass. Agricultural biomass may be generated from energy crops (such as switch grass) grown on marginal lands and cultivated expressly to be used as an energy source, or the biomass may consist of agricultural byproducts such as husks, stovers, foliage, and the like.

Lignocellulosic biomass is generally converted to fungible liquid fuels through either a biochemical or thermochemical process. Biochemical conversion involves pre-treating the biomass, converting the biomass to sugars and then converting the sugars to fuels via fermentation. Thermochemical conversion involves thermal decomposition in the presence of limited oxygen (gasification) or in the absence of oxygen (pyrolysis).

During the pyrolysis or gasification processes, a biomass feedstock is injected into a bio-fuel reactor where the thermochemical conversion occurs. However, the biomass injection process is problematic. Operating temperatures within bio-fuel reactors range between 500° C. (pyrolysis) and 1,200° C. (gasification). As the biomass feedstock approaches the reactor, the biomass particles begin to aggregate and adhere to the injector components. When the partially aggregated fuel is injected into the reactor body it exhibits less than ideal thermal characteristics. Other aspects of reactor design also affect the ability to control pressure within the reactor body.

The need exists for a fast pyrolysis/gasifier injector design that ensures that biomass is efficiently injected into a bioreactor body so that the effectiveness and efficiency of the thermal conversion process is optimized. The current invention comprises an injection system whereby the reactor feedstock is supplemented with nitrogen gas so that clean and efficient pyrolysis (or gasification in alternative embodiments) can occur within the reactor body. The current invention also comprises a fluidized bed gas distributor plate that minimizes the gas pressure drop across the plate so that the pressure within the body of the reactor is more easily and accurately controlled.

### SUMMARY OF THE INVENTION

The current invention is directed to a bio-fuel injection system. The system comprises a bioreactor feedstock and a non-oxygen feedstock gas that supplements the feedstock. The gas-supplemented feedstock is injected into the bioreactor. In the preferred embodiment, the feedstock gas is nitrogen and the bioreactor is a pyrolyzer.

The current invention is also directed to a method of injecting feedstock into a bioreactor. An agricultural feedstock is directed through a metering feeder and into an intermediate tube. The feedstock in the intermediate tube is supplemented with a feedstock gas and then deposited into a fast injection auger. The auger injects the supplemented feedstock into a fluidized bed within the reactor. The fluidized bed is created by directing a fluidization gas through a plurality of screens and into a bed material, thereby fluidizing the bed material.

The current invention is further directed to a fluidization system that includes a gas distributor plate comprised of a plurality of screens. The distributor plate top screen has mesh openings small enough to prevent a bed material from passing through the top screen. The distributor plate bottom screen has mesh openings that are larger than the openings in the top screen. The distributor plate at least partially supports a bed material. The bed material is fluidized by directing a fluidizing gas through the distributor plate and into the bed material.

### BRIEF DESCRIPTION OF THE DRAWINGS

FIG. 1 is a schematic of a conventional prior art fast pyrolysis system.

FIG. 2 is a schematic of the fuel injection system of the current invention.

FIG. 3 is a cross sectional schematic of the gas distributor plate of the current invention.

FIG. 4 is an expanded cross section of the gas distributor plate.

FIG. 5 is a top view of the top screen.

FIG. 6 is a top view of the bottom screen.

### DETAILED DESCRIPTION OF PREFERRED EMBODIMENTS

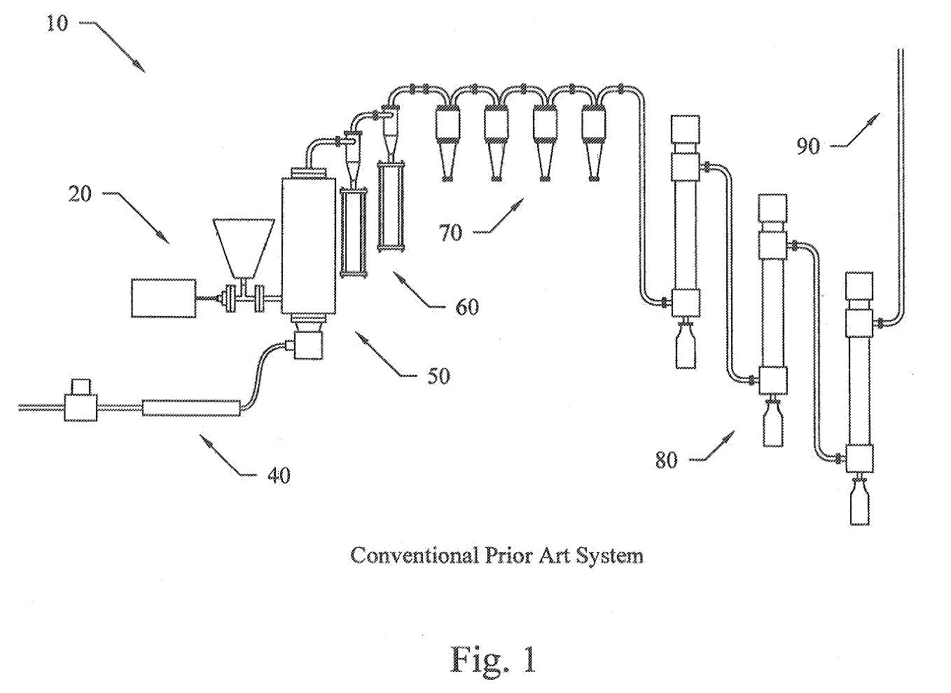

FIG. 1 generally shows a conventional pyrolysis/gasifier system 10. In accordance with conventional designs, a feedstock is fed into a reactor body 50 via a feedstock injection system 20. Simultaneously a gas is injected into the reactor body 50 base via a fluidization gas injection system 40. Pyrolysis/gasification occurs within the reactor body 50 and a pyrolysis/gasification product (bio-syngas, vapor, and char) is produced from an upper portion of the reactor body 50. The product is directed to a particle separation system 60 which removes entrained char and other solid particulate matter from the gas. The remaining gas (condensable vapors and syngas) is then directed to a condenser system 70 which removes liquids from the syngas. Some systems 10 also employ an additional electrostatic precipitator system 80 to further to dry and/or clean the gas. The non-condensable bio-syngas is then directed away from the precipitators 80 via a syngas conduit 90. Some systems re-circulate at least a portion of the syngas to directly or indirectly heat the reactor body 50 during the pyrolysis/gasification process.

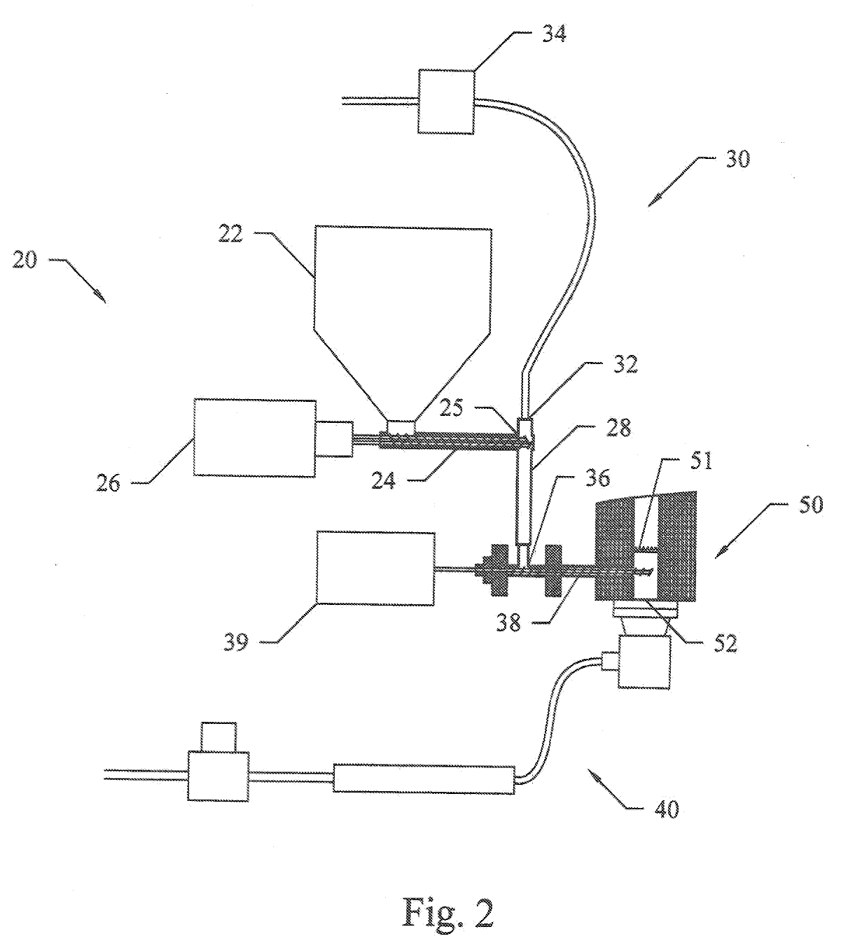

The current invention comprises a novel method and apparatus for improving the feedstock injection system 20 and fluidization gas injection system 40, and thereby improving the precision and efficiency of the gasification/pyrolysis process. FIG. 2 generally shows the feedstock injection system 20 of the current invention. In accordance with the current invention, pre-processed agricultural feedstock (such as husks, stovers, foliage and other agriculturally-generated products) is fed into a vertical hopper 22 which supplies a steady stream of processed feedstock to a twin screw metering feeder 24. FIG. 2 shows a sectional schematic of the twin screw metering feeder 24. In the preferred embodiment, the feeder 24 is powered by an electric power source 26 and the feedstock is “pre-processed” by grinding and/or pulverizing the feedstock into a powder.

The pre-ground feedstock is then fed to an intermediate tube 28 through a feed portal 25. In the preferred embodiment, the intermediate tube is a drop-tube 28. Feedstock accumulates vertically in the drop tube 28. A feedstock gas injection system 30 injects a supplemental gas into the drop tube 28 through a gas injection port 32. In the preferred embodiment, the supplemental feedstock gas is nitrogen and the gas injection port 32 is positioned vertically above the feeder portal 25. In the preferred embodiment, the nitrogen is supplied at a rate proportional to the feedstock flow rate and at a temperature of approximately 23° C. The feedstock gas injection system 30 comprises at least a feedstock gas supply source (not shown) and a pressure regulator 34.

As shown in FIG. 2, the gas-supplemented feedstock is fed through a drop-tube portal 36 into a horizontally oriented fast injection auger 38. FIG. 2 shows a sectional schematic of the fast injection auger 38. In the preferred embodiment, the fast injection auger 38 is powered by an electrical power source 39. The gas-supplemented feedstock is then injected into the body of the reactor 50. In the preferred embodiment, the feedstock is injected into a fluidized bed portion 51 of the reactor body 50. A partial sectional view of the reactor body 50 is shown in FIG. 2.

In the preferred embodiment, a fluidization gas injection system 40 supplies nitrogen to the reactor body 50 as a fluidizing gas medium so that pyrolysis occurs in a nitrogen environment. Consequently the feedstock injection gas supplied by the feedstock gas system 30 has essentially the same composition as the fluidization gas supplied by the fluidization gas injection system 40. Since pyrolysis occurs in a nitrogen environment within the reactor body 50, no dilution or complicating effects are realized by supplementing the feedstock with the nitrogen gas.

Supplementing the feedstock material with nitrogen cools the feedstock and also facilitates the flow of the feedstock through the drop tube 28 and fast injection augur and into the reactor body 50. The supplemental nitrogen keeps the feedstock below the feedstock melting point and deters particles of the feedstock material from adhering to each other (i.e. agglomeration) and deters the feedstock from adhering to the fast injection auger 38. The outward flow of pressurized nitrogen from the fast injection auger 38 also prevents the blow-back of reactor body gasses and fluidized bed material into the auger mechanism 38.

Although both the feedstock and fluidization gases of the preferred embodiment are comprised essentially of nitrogen, in alternative embodiments, one or both of these gases may be comprised of other oxygen or non-oxygen gases. For the purposes of this disclosure, air, steam, or other gases with greater than three percent oxygen are considered to be an “oxygen gas”. Gases that contain less than 3 percent oxygen are considered non-oxygen gases.

In operation, as best shown in FIG. 2, pre-ground feedstock is loaded into a hopper 22 which directs the feedstock into a twin screw metering feeder 24. The feeder 24 conveys the feedstock into a drop tube 28 through a drop tube portal 25. In the drop tube 28, the feedstock is supplemented by a nitrogen feedstock gas. The drop tube 28 subsequently deposits the feedstock into a fast injection auger 38, which injects the supplemented feedstock into the reactor body 50.

Further, as generally shown in FIG. 2, the current invention is also directed to a gas distributor plate 52 positioned in the lower portion of the reactor body 50. The gas distributor plate 52 generally supports a fluidized bed material 51 disposed above the gas distributor plate 52 and distributes fluidizing gas. In some reactors 50, the fluidized bed material 51 is comprised of sand or silica that is “fluidized” as the fluidization gas injection system 40 injects fluidization gas upwardly through the bed material 51. In other reactors, the bed material 51 may be comprised of a catalyst formed into sand-like nodules that can be “fluidized” in a manner similar to sand. The size of the sand or catalyst nodules is typically in the 800 micron range.

Prior art designs of the gas distributor plate 52 typically comprise either a “bubble cap” design or a single structurally robust unitary plate with a plurality of apertures. The bubble cap design comprises a solid plate with multiple rounded “bubble caps” or projections that extend upwardly from the plate and into the bed material. Gas is injected through the bubble caps and into the bed material 51. Specifically, the bubble caps direct the injected gas vertically, horizontally, and diagonally into the bed material so that the bed material is fluidized. Both of the prior art plate designs are typically positioned similar to the gas distributor plate 52 of the current invention.

However, with both the bubble cap and the single unitary plate designs, there is a significant pressure drop across the gas distributor plate 52 when fluidization gas is injected into the reactor body 50. This pressure drop significantly complicates the process of controlling gas volume and pressure within a reactor body 50.

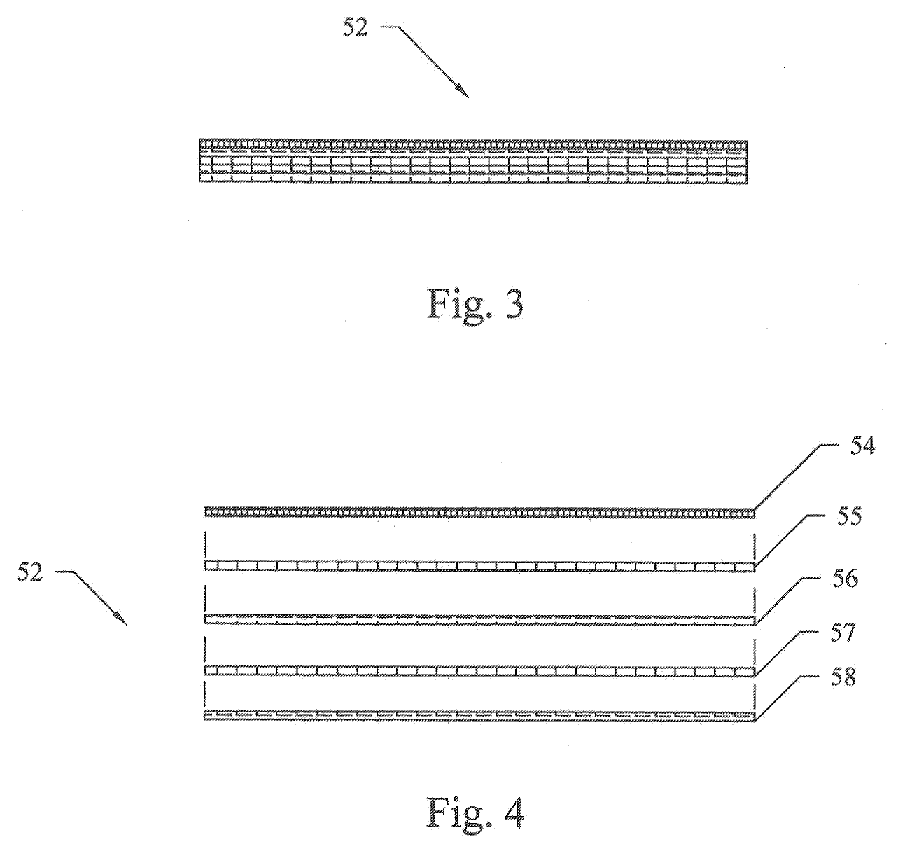

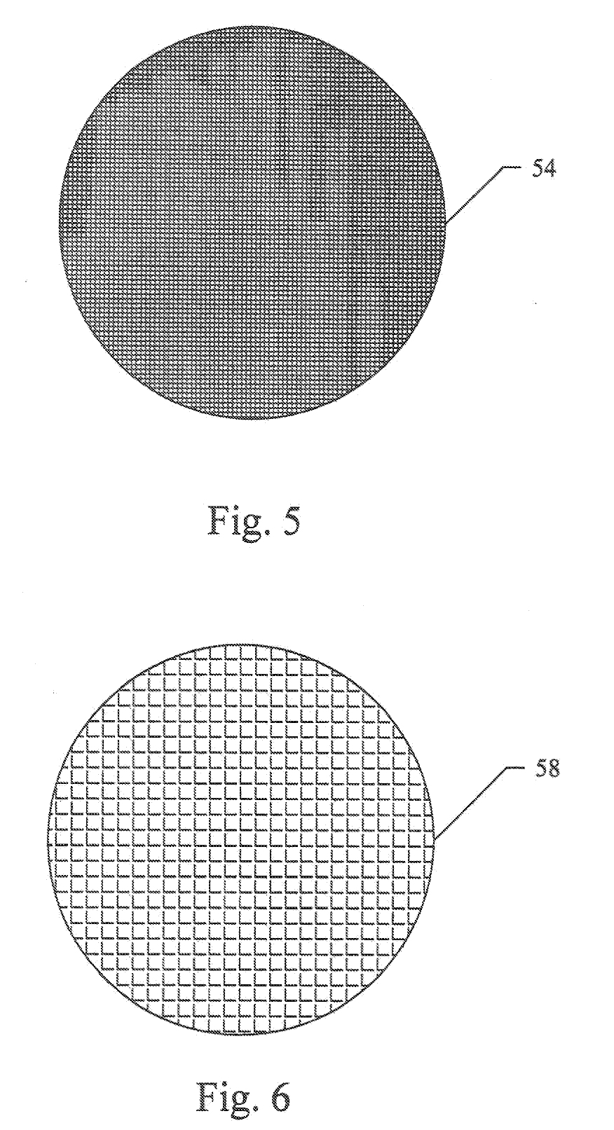

As shown in FIGS. 3-6, the gas distributor plate 52 of the current invention is comprised of plurality (a stack) of fluidizing screens 54, 55, 56, 57, 58 sintered together into a composite gas distribution “plate” 52. The bottom screen 58 is relatively coarse and generally supports the weight of the other screens and the bed: Intermediate screens 55 and 57 are relatively fine, promoting even distribution of the fluidizing gas. Screen 56 is somewhat coarser than screens 55 and 57 providing spacing between them and contributing to the strength of the composite screen stack. The top screen 54 is in contact with the fluidized bed and is similar to screen 56. Screen 54 at least partially protects the fine screen 55 from the abrasiveness of the bed material.

In the preferred embodiment, the first (top) screen 54 is comprised of stainless steel wire having a 100 μm diameter and spaced 250 μm centerline to centerline. The top screen is woven in a standard square weave. The second 55 and fourth 57 screens have similar construction designs that are similar to each other. These screens are comprised of stainless steel wire of two diameters. The wires in one direction of the weave (weft) are 70 μm in diameter and are spaced 82 μm centerline to centerline. The wires woven perpendicular to them (warp) are 50 μm in diameter and are spaced 100 μm centerline to centerline in a “dutch weave” style commonly used in wire filter cloth applications The third screen 56 is similar to the top screen 55 in sizing, spacing and weave style. The bottom screen is shown in FIG. 6. In the preferred embodiment, the fourth (bottom) screen 58 is comprised of stainless steel wire in a dutch weave design. The warp wires are 410 μm diameter closely spaced 205 μm centerline to centerline. The weft wires are 500 μm diameter spaced 1.58 mm centerline to centerline.

The stacked screen design of the current invention results in a very low pressure drop that is essentially negligible, particularly relative to the pressure drop associated with the prior art distributor plate designs, or the pressure drop induced by the fluidizing material 51. The current pressure plate design allows a reactor operator relatively precise control of the fluidizing process and the thermal environment within the reactor body 50. The ability to precisely control the environment within the reactor body 50 greatly increases the flexibility of an operator to use alternative feedstock materials, particularly materials with relatively low melting points such as soy straw, corn stover, lignin, etc. Precise control of the conditions inside the reactor body 50 also significantly increases the efficiency and production potential of the overall pyrolysis/gasification system 10.

For the foregoing reasons, it is clear that the invention provides an innovative feedstock injection system 20 and a novel gas distributor plate 52 which significantly increases the flexibility and efficiency of a pyrolysis or gasification system and process. The invention may be modified in multiple ways and applied in various technological applications which are known to those with skill in the art. For example, although the feedstocks discussed in the disclosure are agriculturally-based, other non-agricultural refuse may also be acceptable as a feedstock.

The current invention may also be modified and customized as required by a specific operation or application, and the individual components may be modified and defined, as required, to achieve the desired result. Although most of the materials of construction are not described, they may include a variety of compositions consistent with the function of the invention. Such variations are not to be regarded as a departure from the spirit and scope of the invention, and all such modifications as would be obvious to one skilled in the art are intended to be included within the scope of the following claims.

What is claimed is:

1. A bio-fuel injection system comprising:
a bioreactor;
a feedstock; and,
a non-oxygen feedstock gas supplementing and pressurizing the feedstock;
wherein the supplemented feedstock is injected into the bioreactor.

2. The injection system of claim 1 wherein a combustible non-oxygen syngas is produced from the bioreactor.

3. The injection system of claim 2 wherein the bioreactor comprises a gasifier or a pyrolyzer.

4. The injection system of claim 1 wherein the feedstock gas comprises nitrogen.

5. The injection system of claim 1 wherein a non-feedstock gas is also injected into the reactor body, the non-feedstock gas comprising a fluidization gas.

6. The injection system of claim 5 wherein the feedstock gas and the fluidization gas have essentially the same composition.

7. The injection system of claim 6 wherein the feedstock gas and the fluidization gas are essentially comprised of nitrogen.

8. The injection system of claim 7 further comprising:
a metering feeder receiving the feedstock from a feedstock source;
an essentially vertical drop tube receiving feedstock from the feeder; and,
a fast injection auger receiving the feedstock and the feedstock gas from the drop tube and injecting the feedstock and the feedstock gas into the reactor.

9. The injection system of claim 7 wherein the feedstock and the feedstock gas are injected into a portion of the reactor comprising a fluidized bed, the fluidized bed comprising a bed material that is fluidized by the fluidization gas.

10. The injection system of claim 9 wherein the bed material comprises nodules.

11. The injection system of claim 9 wherein the bed material comprises sand or silica.

12. The injection system of claim 9 wherein the bed material comprises a catalyst.

13. The injection system of claim 9 further comprising a gas distributor plate, the gas distributor plate at least partially supporting the bed material.

14. The injection system of claim 13 wherein the gas distributor plate comprises a plurality of screens.

15. The injection system of claim 15 wherein at least one of the screens has a dutch weave design.

16. An injection system comprising:
a bed material;
a gas distributor plate at least partially supporting the bed material, the gas distributor plate comprising a plurality of screens; and
a fluidizing gas;
wherein the fluidizing gas is directed through the gas distributor plate so that the bed material is fluidized.

17. The injection system of claim 16 wherein the gas distributor plate is comprised of a first screen and a third screen that have a tighter mesh than a second screen and a fourth screen.

18. The injection system of claim 16 wherein the screens comprising the distributor plate are comprised of stainless steel, at least one of the screens having a dutch weave design.

19. A method of injecting feedstock into a bioreactor, the method comprising the steps of supplementing a feedstock with a non-oxygen gas and injecting the supplemented feedstock into a bioreactor fluidized bed.

20. The method of claim 19 wherein the non-oxygen gas is nitrogen.
